SEQUENCE LISTING (1 sequence)

SEQ ID NO: 1 - DNA, 42 bp, Artificial Sequence
  misc_feature (1)..(42): n = hexaethyleneglycol-aminodT- hexaethyleneglycol
tcgactgctgaaaagcgtcggctggtnaccagccgacgcttt

(19) United States
(12) Patent Application Publication
(10) Pub. No.: US 2003/0022207 A1
(43) Pub. Date: Jan. 30, 2003

(54) Arrayed polynucleotides and their use in genome analysis

(75) Inventors: Shankar Balasubramanian, Nr Saffron Walden (GB); David Klenerman, Nr Saffron Walden (GB); Colin Barnes, Nr Saffron Walden (GB)

Correspondence Address: PALMER & DODGE, LLP, KATHLEEN M. WILLIAMS, 111 HUNTINGTON AVENUE, BOSTON, MA, 02199, US

(73) Assignee: Solexa, Ltd.

(21) Appl. No.: 10/153,240

(22) Filed: May 22, 2002

Related U.S. Application Data
(63) Continuation-in-part of application No. PCT/GB02/00439, filed on Jan. 30, 2002.
(63) Continuation-in-part of application No. 09/771,708, filed on Jan. 30, 2001.
Application No. 09/771,708 is a continuation-in-part of application No. PCT/GB99/02487, filed on Jul. 30, 1999.

(30) Foreign Application Priority Data
Oct. 16, 1998 (GB) GB9822670.7

(51) Int. Cl. C12Q 1/68; C12M 1/34

(52) U.S. Cl. 435/6; 435/287.2

(57) ABSTRACT

The invention encompasses a method for determining a single nucleotide polymorphism present in a genome, comprising: (a) immobilizing polynucleotide molecules onto the surface of a support to form an array comprising polynucleotides located at addresses capable of interrogation, wherein each address of at least a subset of addresses on the array corresponds to a single polynucleotide molecule, and the array permits the subset of addresses to be individually resolved by optical microscopy, and wherein each such single polynucleotide molecule comprises a portion that is immobilized by covalent bonding to the surface and a portion that is capable of interrogation; (b) interrogating an address that corresponds to a single polynucleotide molecule to identify nucleotide sequence in the single polynucleotide molecule; and (c) comparing the nucleotides identified in step (b) with a known consensus sequence, and thereby determining differences between the consensus sequence and the sequence of the single polynucleotide molecule.

### RELATED APPLICATIONS

This application is a continuation-in-part of International Application No. PCT/GB02/00439, filed Jan. 30, 2002, which designated the United States and will be published in English, and which, along with the present application, is a continuation-in-part of application Ser. No. 09/771,708, filed Jan. 30, 2001, which is a continuation-in-part of International Application No. PCT/GB99/02487, which designated the United States and was filed on Jul. 30, 1999, was published in English, and which claims the benefit of British Application GB9822670.7, filed Oct. 16, 1998, and also claims benefit of European Application EP98306094.8, filed Jun. 30, 1998. The entire teachings of the above applications are incorporated herein by reference.

### FIELD OF THE INVENTION

This invention relates to fabricated arrays of polynucleotides, and to their analytical applications. In particular, this invention relates to the use of fabricated polynucleotide arrays in methods for obtaining genetic sequence information.

### BACKGROUND OF THE INVENTION

Advances in the study of molecules have been led, in part, by improvement in technologies used to characterise the molecules or their biological reactions. In particular, the study of nucleic acids, DNA and RNA, has benefited from developing technologies used for sequence analysis and the study of hybridisation events.

An example of the technologies that have improved the study of nucleic acids, is the development of fabricated arrays of immobilised nucleic acids. These arrays typically consist of a high-density matrix of polynucleotides immobilised onto a solid support material. Fodor et al., Trends in Biotechnology (1994) 12:19-26, describes ways of assembling the nucleic acid arrays using a chemically sensitised glass surface protected by a mask, but exposed at defined areas to allow attachment of suitably modified nucleotides. Typically, these arrays can be described as “many molecule” arrays, as distinct regions are formed on the solid support comprising a high density of one specific type of polynucleotide.

An alternative approach is described by Schena et al., Science (1995) 270:467-470, where samples of DNA are positioned at predetermined sites on a glass microscope slide by robotic micropipetting techniques. The DNA is attached to the glass surface along its entire length by non-covalent electrostatic interactions. However, although hybridisation with complementary DNA sequences can occur, this approach may not permit the DNA to be freely available for interacting with other components such as polymerase enzymes, DNA-binding proteins etc.

Recently, the Human Genome Project generated a draft of the entire sequence of the human genome— all 3×10⁹bases. The sequence information represents that of an average human. However, there is still considerable interest in identifying differences in the genetic sequence between different individuals. The most common form of genetic variation is single nucleotide polymorphisms (SNPs). On average one base in 1000 is a SNP, which means that there are 3 million SNPs for any individual. Some of the SNPs are in coding regions and produce proteins with different binding affinities or properties. Some are in regulatory regions and result in a different response to changes in levels of metabolites or messengers. SNPs are also found in non-coding regions, and these are also important as they may correlate with SNPs in coding or regulatory regions. The key problem is to develop a low cost way of determining one or more of the SNPs for an individual.

The nucleic acid arrays can be used to determine SNPs, and they have been used to study hybridisation events (Mirzabekov, Trends in Biotechnology (1994) 12:27-32). Many of these hybridisation events are detected using fluorescent labels attached to nucleotides, the labels being detected using a sensitive fluorescent detector, e.g. a charge-coupled detector (CCD). The major disadvantages of these methods are that it is not possible to sequence long stretches of DNA, and that repeat sequences can lead to ambiguity in the results. These problems are recognised in Automation Technologies for Genome Characterisation, Wiley-Interscience (1997), ed. T. J. Beugelsdijk, Chapter 10: 205-225.

In addition, the use of high-density arrays in a multi-step analysis procedure can lead to problems with phasing. Phasing problems result from a loss in the synchronisation of a reaction step occurring on different molecules of the array. If some of the arrayed molecules fail to undergo a step in the procedure, subsequent results obtained for these molecules will no longer be in step with results obtained for the other arrayed molecules. The proportion of molecules out of phase will increase through successive steps and consequently the results detected will become ambiguous. This problem is recognised in the sequencing procedure described in U.S. Pat. No. 5,302,509. This method is therefore not suitable for the determination of SNPs, where the precise identification of a particular sequence is required.

WO-A-96/27025 is a general disclosure of single molecule arrays. Although sequencing procedures are disclosed, there is little description of the applications to which the arrays can be applied. There is also only a general discussion on how to prepare the arrays.

### SUMMARY OF THE INVENTION

The invention encompasses a method for determining a single nucleotide polymorphism present in a genome, comprising: (a) immobilizing polynucleotide molecules onto the surface of a solid support to form an array comprising polynucleotides located at addresses capable of interrogation, wherein each address of at least a subset of addresses on the array corresponds to a single polynucleotide molecule, and the array permits the subset of addresses to be individually resolved by optical microscopy, and wherein each such single polynucleotide molecule comprises a first portion that is immobilized by covalent bonding to the surface and a second portion that is capable of interrogation; (b) interrogating an address that corresponds to a single polynucleotide molecule to identify nucleotides of a sequence in the single polynucleotide molecule on the array; and (c) comparing the nucleotides identified in step (b) with a known consensus sequence, and thereby determining differences between the consensus sequence and the sequence of the single polynucleotide molecule.

In one embodiment, the polynucleotide molecules comprise fragments of a genome.

In another embodiment, the interrogating step comprises identifying nucleotides of a sequence in the second portion of the single polynucleotide molecule.

In another embodiment, step (b) comprises: (i) contacting the array with each of the nucleotides dATP, dTTP, dGTP and dCTP, under conditions that permit a nucleic acid polymerase reaction to proceed and thereby form sequences complementary to the polynucleotides immobilized on said array; (ii) determining the incorporation of a nucleotide in the complementary sequences formed in step (i); and (iii) optionally repeating the steps (i) and (ii).

In a preferred embodiment, each nucleotide contains a removable fluorescent label.

In another preferred embodiment, each nucleotide contains a removable blocking group that prevents further nucleotide incorporation, and the blocking group is removed after each step of determining nucleotide incorporation.

In another embodiment, step (i) is carried out by first contacting the array with three of the four nucleotides dATP, dTTP, dCTP and dGTP under conditions that permit a nucleic acid polymerase reaction to proceed and thereby form sequences complementary to those in the array, then removing unincorporated nucleotides from the array, and then contacting the array with the remaining nucleotide under conditions that permit a nucleic acid polymerase reaction to proceed and thereby form sequences complementary to those in the array, so that step (ii) proceeds only after incorporation of said remaining nucleotide.

In another embodiment, adjacent single polynucleotides of the array are separated by a distance of at least 10 nm.

In another embodiment, the adjacent single polynucleotides are separated by a distance of at least 100 nm.

In another embodiment, the adjacent single polynucleotides are separated by a distance of at least 250 nm.

In another embodiment, the array has a density of from 10⁶ to 10⁹ single polynucleotides per cm².

In another embodiment, the array density is from 10⁷ to 10⁹ single polynucleotides per cm².

In another embodiment the polynucleotides are immobilised to the solid support via the 5′ terminus, the 3′ terminus or via an internal nucleotide.

According to one aspect of the invention, a method for determining a single nucleotide polymorphism present in a genome comprises the steps of: (i) immobilising fragments of the genome onto the surface of a solid support to form an array of polynucleotide molecules capable of interrogation, wherein the array allows the molecules to be individually resolved by optical microscopy, and wherein each molecule is immobilised by covalent bonding to the surface, other than at that part of each molecule that can be interrogated; (ii) identifying nucleotides at selected positions in the genome; and (iii) comparing the results of step (ii) with a known consensus sequence, and identifying any differences between the consensus sequence and the genome.

The features or addresses of the arrays of the present invention comprise what are effectively single molecules. This has many important benefits for the study of the molecules and their interaction with other biological molecules. In particular, fluorescent labels can be used in interactions with the single polynucleotide molecules and can be detected using an optical microscope linked to a sensitive detector, resulting in a distinct signal for each polynucleotide.

The arrays permit a massively parallel approach to monitoring fluorescent or other events on the polynucleotides. Such massively parallel data acquisition makes the arrays extremely useful in the detection and characterisation of single nucleotide polymorphisms.

As used herein, the term “feature,” or the equivalent term “address,” refers to each nucleic acid molecule occupying a discrete physical location on an array; if a given sequence is represented at more than one such site, each site is classified as a feature. It is preferred that a subset of the features on an array according to the invention comprise a single polynucleotide molecule only. It is more preferred that substantially all of the features on an array according to the invention comprise a single polynucleotide molecule only. As used herein, “substantially all of the features” means at least 50%, and preferably at least 60%, 70%, 80%, 85%, 90%, 92%, 94%, 96%, 98%, 99% or more of the features.

As used herein, the term “array” refers to a population of nucleic acid molecules that is distributed over a solid support; preferably, these molecules differing in sequence are spaced at a distance from one another sufficient to permit the identification of discrete addresses or features of the array. The population can be a heterogeneous mixture of nucleic acid molecules.

“Solid support”, as used herein, refers to the material to which a nucleic acid sample is attached. Suitable solid supports are available commercially, and will be apparent to the skilled person. The supports can be manufactured from materials such as glass, ceramics, silica and silicon. Supports with a gold surface may also be used. The supports usually comprise a flat (planar) surface, or at least a structure in which the polynucleotides to be interrogated are in approximately the same plane. Alternatively, the solid support can be non-planar, e.g., a microbead. Any suitable size may be used. For example, the supports might be on the order of 1-10 cm in each direction.

As used herein, the term “interrogate” means contacting the arrayed polynucleotide molecule with any other molecule, wherein the physical interaction provides information regarding a characteristic of the arrayed polynucleotide. The contacting can involve covalent or non-covalent interactions with the other molecule. As used herein, “information regarding a characteristic” means information regarding the sequence of one or more nucleotides in the polynucleotide, the length of the polynucleotide, the base composition of the polynucleotide, the Tₘ of the polynucleotide, the presence of a specific binding site for a polypeptide or other molecule, the presence of an adduct or modified nucleotide, or the three-dimensional structure of the polynucleotide.

As used herein, the term “features capable of interrogation” or “addresses capable of interrogation” refers to array features or addresses in which the immobilized single polynucleotide comprises at least a portion that is accessible for a physical interaction with another molecule or molecules, wherein the interaction provides information regarding a characteristic of the arrayed polynucleotide. For example, when nucleic acid sequence information is the characteristic sought to be determined, features capable of interrogation include those features wherein at least a portion of the immobilized single polynucleotide molecule is physically accessible to and can serve as a functional substrate for a nucleic acid polymerase enzyme. By “functional substrate” is meant that the immobilized polynucleotide itself, or a primer annealed to it, can be extended by the template-dependent polymerase activity of such enzyme.

As used herein, the term “single polynucleotide molecule” refers to one molecule of a nucleic acid sequence. Thus, an array feature or address corresponding to a single polynucleotide molecule consists of one polynucleotide molecule immobilized at that location on a solid support. This is in contrast to the array features of the prior art, in which a given feature or address typically comprises a plurality of copies of a given nucleic acid molecule, often thousands of copies or more.

“Single polynucleotide molecules” according to the invention can be single- or double-stranded. In one embodiment, the single polynucleotide molecule is single stranded. In another embodiment, the single polynucleotide molecule to be interrogated is a single nucleic acid strand attached to the array by hybridization to a covalently immobilized oligonucleotide; in this embodiment, the molecule to be interrogated is still considered to be a “single polynucleotide molecule.” In another embodiment, single polynucleotide molecules on the array are single stranded, yet form a hairpin at the immobilized end.

As used herein, the term “individually resolved” is used to indicate that, when visualised, it is possible to distinguish one polynucleotide on the array from its neighbouring polynucleotides. Visualisation may be effected by the use of reporter labels, e.g. fluorophores, the signal of which is individually resolved. Visualisation can be accomplished through the use of optical microscopy methods known in the art.

The terms “arrayed polynucleotides” and “polynucleotide arrays” are used herein to define a plurality of single polynucleotides. The term is intended to include the attachment of other molecules to a solid surface, the molecules having a polynucleotide attached that can be further interrogated during the SNP analysis. For example, the arrays can comprise linker molecules immobilised on a solid surface, the linker molecules being conjugated or otherwise bound to a polynucleotide that can be interrogated, to determine the presence of a SNP.

As used herein, the term “portion that is immobilized by bonding to the surface” refers to the nucleotide or nucleotides of an immobilized single polynucleotide molecule that is or are either directly involved in linkage to the solid substrate, or, because of their proximity to the point of immobilization, are not physically accessible to be capable of interrogation (e.g., to serve as a template or substrate for the primer extension activity of a nucleic acid polymerase enzyme). Depending upon the means of immobilization (e.g., direct immobilization, immobilization through a linker, etc.), the portion of a polynucleotide that is immobilized by bonding to a surface can be as small as one nucleotide or as large as 100 nucleotides or more, as long as there remains at least a portion of the immobilized polynucleotide molecule that is capable of interrogation. It is preferred that polynucleotides be immobilized by either their 5′ end or their 3′ end, but polynucleotides can also be immobilized via an internal nucleotide.

As used herein, the term “portion that is capable of interrogation” refers to that portion of an immobilized single polynucleotide molecule that is physically accessible to a physical interaction with another molecule or molecules, the interaction of which provides information regarding a characteristic of the arrayed polynucleotide as defined herein. Generally, the “portion of an immobilized single polynucleotide molecule that is capable of interrogation” is that part which is not the “portion that is immobilized by bonding to the surface” as that term is defined herein.

As used herein, the term “blocking group” refers to a moiety attached to a nucleotide which, while not interfering substantially with template-dependent enzymatic incorporation of the nucleotide into a polynucleotide chain, abrogates the ability of the incorporated nucleotide to serve as a substrate for further nucleotide addition. A “removable blocking group” is a blocking group that can be removed by a specific treatment that results in the cleavage of the covalent bond between the nucleotide and the blocking group. Specific treatments can be, for example, a photochemical, chemical or enzymatic treatment that results in the cleavage of the covalent bond between the nucleotide and the fluorescent label. Removal of the blocking group will restore the ability of the incorporated, formerly blocked nucleotide to serve as a substrate for further enzymatic nucleotide additions.

As used herein, the term “removable fluorescent label” refers to a covalently linked fluorescent label on a nucleotide, which label can be removed by a specific treatment of the nucleotide or a polynucleotide comprising the nucleotide. Specific treatments can be, for example, a photochemical, chemical or enzymatic treatment that results in the cleavage of the covalent bond between the nucleotide and the fluorescent label. In those instances where the fluorescent label blocks further nucleotide incorporation, removal of the fluorescent label after incorporation of the labeled nucleotide restores the ability of the formerly labeled nucleotide to serve as a substrate for further enzymatic nucleotide additions.

As used herein, the phrase “conditions that permit a nucleic acid polymerase reaction to proceed and thereby form sequences complementary to the polynucleotides immobilized on the array” refers to those refers to those conditions of salt concentration (metallic and non-metallic salts), pH, temperature, and necessary cofactor concentration under which a given polymerase enzyme catalyzes the extension of an annealed primer. Conditions for the primer extension activity of a wide range of polymerase enzymes are known in the art. As one example, conditions permitting the extension of a nucleic acid primer by Klenow exo-polymerase include the following: 50 mM Tris.HCl, 1 mM EDTA, 5 mM MgCl₂, 10 mM NaCl (pH 7.4), 2 μM dNTPs, 1 mM DTT, Klenow exo- (10 units in 100 μl final volume) at 37° C. A chain terminator can be included, depending upon the type of primer extension or sequencing being performed.

### DETAILED DESCRIPTION OF THE INVENTION

According to the present invention, the single polynucleotides immobilised onto the surface of a solid support should be capable of being resolved by optical means. This means that, within the resolvable area of the particular imaging device used, there must be one or more distinct signals, each representing one polynucleotide. Typically, the polynucleotides of the array are resolved using a single molecule fluorescence microscope equipped with a sensitive detector, e.g. a charge-coupled device (CCD). Each polynucleotide of the array can be analysed simultaneously or, by scanning the array, a fast sequential analysis can be performed.

The polynucleotides of the array are preferably derived from fragments of genomic DNA.

The density of the array is not critical. However, the present invention can make use of a high density of single molecules (polynucleotides), and these are preferable. For example, arrays with a density of 10⁶ to 10⁹ single polynucleotides per cm² can be used. Preferably, the density is at least 10⁷/cm² to 10⁹/cm². These high density arrays are in contrast to other arrays which may be described in the art as “high density” but which are not necessarily as high and/or which do not allow single molecule resolution. On a given array, it is the number of single polynucleotides, rather than the number of features, that is important. The concentration of nucleic acid molecules applied to the support can be adjusted in order to achieve the highest density of addressable single polynucleotide molecules. At lower application concentrations, the resulting array will have a high proportion of addressable single polynucleotide molecules at a relatively low density per unit area. As the concentration of nucleic acid molecules is increased, the density of addressable single polynucleotide molecules will increase, but the proportion of single polynucleotide molecules capable of being addressed will actually decrease. One skilled in the art will therefore recognize that the highest density of addressable single polynucleotide molecules can be achieved on an array with a lower proportion or percentage of single polynucleotide molecules relative to an array with a high proportion of single polynucleotide molecules but a lower physical density of those molecules.

Using the methods and apparatus of the present invention, it can be possible to image at least 10⁷ or 10⁸ polynucleotides. Fast sequential imaging can be achieved using a scanning apparatus; shifting and transfer between images can allow higher numbers of molecules to be imaged.

The extent of separation between the individual polynucleotides on the array will be determined, in part, by the particular technique used to resolve the individual polynucleotide. Apparatus used to image molecular arrays are known to those skilled in the art. For example, a confocal scanning microscope can be used to scan the surface of the array with a laser to image directly a fluorophore incorporated on the individual molecule by fluorescence. Alternatively, a sensitive 2-D detector, such as a charge-coupled device, can be used to provide a 2-D image representing the individual polynucleotides on the array.

Resolving single polynucleotides on the array with a 2-D detector can be done if, at 100× magnification, adjacent polynucleotides are separated by a distance of approximately at least 250 nm, preferably at least 300 nm and more preferably at least 350 nm. It will be appreciated that these distances are dependent on magnification, and that other values can be determined accordingly, by one of ordinary skill in the art.

Other techniques such as scanning near-field optical microscopy (SNOM) are available which are capable of greater optical resolution, thereby permitting more dense arrays to be used. For example, using SNOM, adjacent polynucleotides can be separated by a distance of less than 100 nm, e.g. 10 nm. For a description of scanning near-field optical microscopy, see Moyer et al., Laser Focus World (1993) 29(10).

An additional technique that can be used is surface-specific total internal reflection fluorescence microscopy (TIRFM); see, for example, Vale et al., Nature, (1996) 380: 451-453). Using this technique, it is possible to achieve wide-field imaging (up to 100 μm×100 μm) with single molecule sensitivity. This can allow arrays of greater than 10⁷ resolvable polynucleotides per cm² to be used.

Additionally, the techniques of scanning tunnelling microscopy (Binnig et al., Helvetica Physica Acta (1982) 55:726-735) and atomic force microscopy (Hansma et al., Ann. Rev. Biophys. Biomol. Struct. (1994) 23:115-139) are suitable for imaging the arrays of the present invention. Other devices which do not rely on microscopy can also be used, provided that they are capable of imaging within discrete areas on a solid support.

Single polynucleotides can be arrayed by immobilisation to the surface of a solid support. This can be carried out by any known technique, provided that suitable conditions are used to ensure adequate separation. Generally the array is produced by dispensing small volumes of a sample containing a mixture of the fragmented genomic DNA onto a suitably prepared solid surface, or by applying a dilute solution to the solid surface to generate a random array. The formation of the array then permits interrogation of each arrayed polynucleotide to be carried out.

Suitable solid supports are available commercially, and will be apparent to the skilled person. The supports can be manufactured from materials such as glass, ceramics, silica and silicon. The supports usually comprise a flat (planar) surface, or an array in which the polynucleotides to be interrogated are in the same plane. However, “solid supports” as the term is used herein can also encompass non-planar supports, for example, a microbead. Any suitable size can be used. For example, the supports might be of the order of 1-10 cm in each direction.

Immobilisation can be by specific covalent or non-covalent interactions. Covalent attachment is preferred. Immobilisation can be at an internal position or at either the 5′ or 3′ position. However, the polynucleotide can be attached to the solid support at any position along its length, the attachment acting to tether the polynucleotide to the solid support. The immobilised polynucleotide is then able to undergo interactions at positions distant from the solid support. Typically the interaction will be such that it is possible to remove any molecules bound to the solid support through non-specific interactions, e.g. by washing. Immobilisation in this manner results in well separated single polynucleotides.

In one embodiment, the array comprises polynucleotides with a hairpin loop structure, one end of which comprises the target polynucleotide derived from the genomic DNA sample.

The term “hairpin loop structure” refers to a molecular stem and loop structure formed from the hybridisation of complementary polynucleotides that are covalently linked. The stem comprises the hybridised polynucleotides and the loop is the region that covalently links the two complementary polynucleotides. Anything from a 5 to 25 (or more) base pair double-stranded (duplex) region can be used to form the stem. In one embodiment, the structure can be formed from a single-stranded polynucleotide having complementary regions. The loop in this embodiment can be anything from 2 or more non-hybridised nucleotides. In a second embodiment, the structure is formed from two separate polynucleotides with complementary regions, the two polynucleotides being linked (and the loop being at least partially formed) by a linker moiety. The linker moiety forms a covalent attachment between the ends of the two polynucleotides. Linker moieties suitable for use in this embodiment will be apparent to the skilled person. For example, the linker moiety can be polyethylene glycol (PEG).

There are many different ways of forming the hairpin structure to incorporate the target polynucleotide. However, a preferred method is to form a first molecule capable of forming a hairpin structure, and ligate the target polynucleotide to this. Ligation can be carried out either prior to or after immobilisation to the solid support. The resulting structure comprises the target polynucleotide at one end of the hairpin and a primer polynucleotide at the other end. The target polynucleotide can be either single stranded or double stranded as long as the 3′-end of the hairpin contains a free hydroxyl amenable to further polymerase extension.

The DNA to be analyzed can be PCR-amplified or used directly to generate fragments of DNA using either restriction endonucleases, other suitable enzymes, a mechanical form of fragmentation or a non-enzymatic chemical fragmentation method or a combination thereof. The DNA can be genomic DNA. The fragments can be of any suitable length, preferably from 20 to 2000 bases, more preferably 20 to 1000 bases, most preferably 20 to 200 bases. In the case of fragments generated by restriction endonucleases, hairpin structures bearing a complementary restriction site at the end of the first hairpin can be used. In the case of non-selective fragmentation, ligation of one strand of the DNA sample fragments can be achieved by various methods.

Method 1: The fragments are ligated to a hairpin made, for example, with a 3′ overhang containing all possible sequences of a few nucleotides (preferably 3-20 bases long, more preferably 5-9 bases long), a 3′ hydroxyl and a 5′ phosphate. Ligation creates a 5′ overhang that is capable of being sequenced from the 3′ hydroxyl of the hairpin using the newly ligated genomic fragment as a template by the methods described.

Method 2: in the design of the hairpin, a single (or more) base gap can be incorporated at the 3′ end (the receded strand) such that upon ligation of the DNA fragment only one strand is covalently joined to the hairpin. The base gap can be formed by hybridising a further separate polynucleotide to the 5′-end of the first hairpin structure. On ligation, the DNA fragment has one strand joined to the 5′-end of the first hairpin, and the other strand joined to the 3′-end of the further polynucleotide. The further polynucleotide (and the other strand of the DNA fragment) can then be removed by disrupting hybridisation.

Method 3: Genomic fragments are left in their double stranded-form or are made to be double stranded and blunt ended by conventional means and are phosphatased to produce 3′ and 5′ hydroxyls as is known in the art. The fragments are ligated to a hairpin made for example with a blunt end, a 3′ hydroxy and a 5′ phosphate. Ligation of only one strand creates a 5′ overhang that is capable of being sequenced from the 3′ hydroxyl of the hairpin using the newly ligated genomic fragment as a template by the methods described.

The net result should be covalent ligation of only one strand of a DNA fragment of genomic DNA, to the hairpin, the DNA fragment being then in the form of a 5′ overhang that is capable of being sequenced. Such ligation reactions can be carried out in solution at optimised concentrations based on conventional ligation chemistry, for example, carried out by DNA ligases or non-enzymatic chemical ligation. Should the fragmented DNA be generated by random shearing of genomic DNA, then the ends can be filled in with any polymerase to generate blunt-ended fragments which can be blunt-end-ligated onto blunt-ended hairpins. Alternatively, the blunt-ended DNA fragments can be ligated to oligonucleotide adapters which are designed to allow compatible ligation with the sticky-end hairpins, in the manner described previously.

The hairpin-ligated DNA constructs can then be covalently attached to the surface of a solid support to generate the single molecule array, or ligation can follow attachment to form the array.

The arrays can then be used in procedures to determine the presence of a SNP. In the case of random fragmentation of the DNA sample, cycles of sequencing can be performed to place the fragment in a unique context within the sample from which it originated. If the target fragments are generated via restriction digest of genomic DNA, the recognition sequence of the restriction or other nuclease enzyme will provide 4, 6, 8 bases or more of known sequence (dependent on the enzyme). Further sequencing of at least 4 bases and preferably between 10 and 30 bases on the array should provide sufficient overall sequence information to place that stretch of DNA into unique context with a total human genome sequence, thus enabling the sequence information to be used for genotyping and more specifically single nucleotide polymorphism (SNP) scoring.

Simple calculations have suggested the following based on sequencing a 10⁷ molecule array prepared from hairpin ligation: for a 6 base pair recognition sequence, a single restriction enzyme will generate approximately 10⁶ ends of DNA. If a stretch of 13 bases is sequenced on the array (i.e. 13×10⁶ bases), approximately 13,000 SNPs will be detected. The approach is therefore suitable for forensic analysis or any other system which requires unambiguous identification of individuals to a level as low as 10³ SNPs.

It is of course possible to sequence the complete target polynucleotide, if required.

Sequencing can be carried out by the stepwise identification of suitably labelled nucleotides, referred to in U.S. Pat. No. 5,654,413 as “single base” sequencing methods. The target polynucleotide is primed with a suitable primer (or prepared as a hairpin construct which will contain the primer as part of the hairpin), and the nascent chain is extended in a stepwise manner by the polymerase reaction. Each of the different nucleotides (A, T, G and C) incorporates a unique fluorophore which can be located at the 3′ position to act as a blocking group to prevent uncontrolled polymerisation. The polymerase enzyme incorporates a nucleotide into the nascent chain complementary to the target, and the blocking group prevents further incorporation of nucleotides. The array surface is then cleared of unincorporated nucleotides and each incorporated nucleotide is “read” optically by a charge-coupled detector using laser excitation and filters. The 3′-blocking group is then removed (deprotected), to expose the nascent chain for further nucleotide incorporation.

Because the array consists of distinct optically resolvable polynucleotides, each target polynucleotide will generate a series of distinct signals as the fluorescent events are detected. Details of the sequence are then determined and can be compared with known sequence information to identify SNPs.

The number of cycles that can be achieved is governed principally by the yield of the deprotection cycle. If deprotection fails in one cycle, it is possible that later deprotection and continued incorporation of nucleotides can be detected during the next cycle. Because the sequencing is performed at the single molecule level, the sequencing can be carried out on different polynucleotide sequences at one time without the necessity for separation of the different sample fragments prior to sequencing. This sequencing also avoids the phasing problems associated with prior art methods.

The labelled nucleotides can comprise a separate label and removable blocking group, as will be appreciated by those skilled in the art. In this context, it will usually be necessary to remove both the blocking group and the label prior to further incorporation.

Deprotection can be carried out by chemical, photochemical or enzymatic reactions. A similar, and equally applicable, sequencing method is disclosed in EP-A-0640146. Other suitable sequencing procedures will be apparent to the skilled person.

It is not necessary to determine the sequence of the full polynucleotide fragment. For example, it can be preferable to determine the sequence of 16-30 specific bases, which is sufficient to identify the DNA fragment by comparison to a consensus sequence, e.g. to that known from the Human Genome Project. Any SNP occurring within the sequenced region can then be identified. The specific bases do not have to be contiguous. For example, the procedure can be carried out by the incorporation of non-labelled bases followed, at pre-determined positions, by the incorporation of a labelled base. Provided that the sequence of sufficient bases is determined, it should be possible to identify the fragment. Again, any SNPs occurring at the determined base positions, can be identified. For example, the method can be used to identify SNPs that occur after cytosine. Template DNA (genomic fragments) can be contacted with each of the bases A, T and G, added sequentially or together, so that the complementary strand is extended up to a position that requires C. Non-incorporated bases can then be removed from the array, followed by the addition of C. The addition of C is followed by monitoring the next base incorporation (using a labelled base). By repeating this process a sufficient number of times, a partial sequence is generated where each base immediately following a C is known. It will then be possible to identify the full sequence, by comparison of the partial sequence to a reference sequence. It will then also be possible to determine whether there are any SNPs occurring after any C.

To further illustrate this, a device can comprise 10⁷ restriction fragments per cm². If 30 bases are determined for each fragment, this means 3×10⁸ bases are identified. Statistically, this should determine 3×10⁵ SNPs for the experiment. The approach therefore permits analysis of large amounts of sequence for SNPs.

The images and other information about the arrays, e.g. positional information, etc. are processed by a computer program which can perform image processing to reduce noise and increase signal or contrast, as is known in the art. The computer program can perform an optional alignment between images and/or cycles, extract the single molecule data from the images, correlate the data between images and cycles and specify the DNA sequence from the patterns of signal produced from the individual molecules.

The individual DNA sequence reads of at least 4 bases, and more preferably at least 16 bases in the case of human genomic DNA, and more preferably 16-30 bases, are aligned and compared with a genomic sequence. The methods for performing this alignment are based upon techniques known to those skilled in the art. The individual DNA sequence reads are aligned with respect to the reference sequence by finding the best match between the individual DNA sequence reads and the reference sequence. Using the known alignments, one or many individual DNA sequence reads covering a given region of the genomic DNA sequence are obtained. All the aligned individual DNA sequence reads are interpreted at each nucleotide position in the reference sequence as either containing the identical sequence to the reference sequence, or containing an error in some of the individual DNA sequence reads, or containing a known or novel mutation, SNP, deletion, insertion, etc. at that position. Furthermore, for most chromosomes, at each position in the reference sequence, the individual can contain one (homozygous) or two (heterozygous) different nucleotides corresponding to the two copies of each chromosome. The sum total of all the individual variations in the reference sequence corresponding to a given individual sample is collectively referred to as a “total genotype”.

The following Example illustrates the invention.

### EXAMPLE

Preparation of hairpin single molecule array (unlabelled DNA): A 10 μM solution of oligonucleotide (5′-TCgACTgCTgAAAAgCgTCggCTggT-HEG-aminodT-HEG-ACCAgCCgACGCTTT; SEQ ID NO. 1) in DMF containing 10% water and 1% diisopropylethylamine (DIPEA) was prepared. To this, a stock solution of the GMBS crosslinker was added to give a final concentration of 1 mM N-[γ-Maleimidobutryloxy]succinimide ester (GMBS) (100 eqvs.). The reaction was left for 1 h at room temperature, purified using a NAP size exclusion column and freeze-dried in aliquots that were re-dissolved immediately prior to use.

A fused silica slide was treated with decon for 12 h then rinsed with water, EtOH, dried and placed in a flow cell. A solution of the GMBS DNA (150 nM) and mercaptopropyltrimethoxysilane (3 μM) in 9:1 sodium acetate (30 mM, pH 4.3): isopropanol was placed over the slide for 30 min. at 65° C. The cell was flushed first with 50 mM Tris.HCl, 1 mM EDTA, pH 7.4 and then 50 mM Tris.HCl, 1 mM EDTA, 5 mM MgCl₂, 10 mM NaCl (pH 7.4) (10 mL) at 37° C. (TKF buffer). The cell was filled with 100 μL of 2 μM Cy5-dCTP, 2 μM dTTP, 2 μm dATP, 1 mM DTT, Klenow exo- (10 units) in TKF buffer and incubated at 37° C. for 10 mins. than flushed with TKF buffer (20 mL) and TKF buffer containing NaCl (1 M) which removes bound protein. A second cycle consisting of 100 μL of 2 μM Cy3-dCTP, 2 μM dGTP, 2 μM dATP, 1 mM DTT, Klenow exo- (10 units) in TKF buffer was incubated at 37° C. for 10 mins. then flushed with TKF buffer (20 mL) and TKF buffer containing NaCl (1 M).

The flowcell was inverted so that the chamber coverslip contacts the objective lens of an inverted microscope (Nikon TE200) via an immersion oil interface. A 60° fused silica dispersion prism was optically coupled to the back of the slide through a thin film of glycerol. Laser light was directed at the prism such that at the glass/sample interface subtended an angle of approximately 68° to the normal of the slide and subsequently underwent Total Internal Reflection (TIR). Fluorescence from the surface produced by excitation with the surface specific evanescent wave generated by TIR was collected by the 100× objective lens of the microscope and imaged onto an intensified charged coupled device (ICCD) camera (Pentamax, Princeton Instruments).

Images were recorded using a combination of a 532 Nd:YAG laser with a 580DF30 emission filter (Omega optics) and a pumped dye laser at 630 nm with a 670DF40 emission filter. Images were recorded with an exposure of 500 ms and maximum camera gain and a laser power of 50 mW (green) and 40 mW (red) at the prism.

Two colour fluorophore labelled nucleotide incorporations were identified by the co-localisation of discreet points of fluorescence from single molecules of Cy3 and Cy5 following superimposing the two images. Molecules were considered co-localised when fluorescent points were within a pixel separation of each other. For a 90 μm and 90 μm field projected onto a CCD array of 512×512 pixels the pixel size dimension is 176 nm.

An average 46.2% of Cy3 and 57.5% of Cy5 were colocalized; showing >50% of the molecules that underwent the Cy5 incorporation underwent a second cycle of Cy3 incorporation. In the absence of enzyme in the second cycle the level of Cy3 was greatly reduced and the colocalisation was <2%. Polymerase fidelity controls, whereby the dATP or dGTP was omitted from the cycles, gave colocalisation levels of approximately 4%.

This demonstrates that sequence determination at the single molecule level can be achieved and makes it possible to extend this to genomic fragments to identify SNPs.

### Other Embodiments

Those skilled in the art should appreciate that they can readily use the disclosed conception and specific embodiments as a basis for designing or modifying other methods for carrying out the same purposes of the present invention without departing from the spirit and scope of the invention as defined by the appended claims. All literature and patent references referred to herein are hereby incorporated by reference in their entirety.

1. A method for determining a single nucleotide polymorphism present in a genome, comprising
(a) immobilizing polynucleotide molecules onto the surface of a solid support to form an array comprising polynucleotides located at addresses capable of interrogation, wherein each address of at least a subset of addresses on the array corresponds to a single polynucleotide molecule, and the array permits said subset of addresses to be individually resolved by optical microscopy, and wherein each said single polynucleotide molecule comprises a first portion that is immobilized by bonding to the surface and a second portion that is capable of interrogation;
(b) interrogating a said address to identify nucleotides of a sequence in a said single polynucleotide molecule on said array; and
(c) comparing the nucleotides identified in step (b) with a known consensus sequence, and thereby determining differences between the consensus sequence and said sequence of said single polynucleotide molecule.

2. The method of claim 1 wherein said polynucleotide molecules comprise fragments of a genome.

3. The method of claim 1 wherein said interrogating comprises identifying nucleotides of a sequence in said second portion of said single polynucleotide molecule.

4. The method of claim 1, wherein step (b) comprises
(i) contacting the array with each of the nucleotides dATP, dTTP, dGTP and dCTP, under conditions that permit a nucleic acid polymerase reaction to proceed and thereby form sequences complementary to the polynucleotides immobilized on said array;
(ii) determining the incorporation of a nucleotide in the complementary sequences formed in step (i); and
(iii) optionally repeating said steps (i) and (ii).

5. The method of claim 4, wherein each nucleotide contains a removable fluorescent label.

6. The method of claim 4, wherein each nucleotide contains a removable blocking group that prevents further base incorporation, and wherein the blocking group is removed after each step of determining nucleotide incorporation.

7. The method of claim 4, wherein step (i) is carried out by first contacting the array with three of the four nucleotides dATP, dTTP, dCTP and dGTP under conditions that permit a nucleic acid polymerase reaction to proceed and thereby form sequences complementary to those in the array, then removing unincorporated nucleotides from the array, and then contacting the array with the remaining nucleotide under conditions that permit a nucleic acid polymerase reaction to proceed and thereby form sequences complementary to those in the array, so that step (ii) proceeds only after incorporation of said remaining nucleotide.

8. The method of any one of claims 1 to 5, wherein adjacent polynucleotides of the array are separated by a distance of at least 10 nm.

9. The method of claim 8, wherein the polynucleotides are separated by a distance of at least 100 nm.

10. The method of claim 8, wherein the polynucleotides are separated by a distance of at least 250 nm.

11. The method of claim 1, wherein the array has a density of from 10⁶ to 10⁹ single polynucleotides per cm².

12. The method of claim 11, wherein the density is from 10⁷ to 10⁹ single polynucleotides per cm².

13. The method of claim 1, wherein said polynucleotides are immobilised to said solid support via the 5′ terminus, the 3′ terminus or via an internal nucleotide.
